(19) United States
(12) Patent Application Publication
(10) Pub. No.: US 2025/0262088 A1
(43) Pub. Date: Aug. 21, 2025

(54) EXTERNAL THERMAL GRILL PELTIER DEVICE AND METHOD TO STIMULATE NERVES AND MUSCLES

(71) Applicant: The Alfred E. Mann Foundation for Scientific Research, Valencia, CA (US)

(72) Inventors: Trent Jonathan Schramm, Castaic, CA (US); Anthony C. Ng, Santa Clarita, CA (US); Neil Hamilton Talbot, La Crescenta, CA (US)

(21) Appl. No.: 19/040,580

(22) Filed: Jan. 29, 2025

Related U.S. Application Data
(60) Provisional application No. 63/554,659, filed on Feb. 16, 2024.

(51) Int. Cl. A61F 7/02; A61F 7/00; A61H 23/02; A61N 5/06; A61N 5/067

(52) U.S. Cl. CPC A61F 7/02; A61H 23/0245; A61N 5/067; A61F 2007/0024; A61F 2007/0075; A61F 2007/0093; A61F 2007/0094; A61F 2007/0096; A61H 2201/10; A61H 2209/00; A61N 2005/0651; A61N 2005/0659; A61N 2005/0663

(57) ABSTRACT

A system configured to alleviate pain includes a thermal device and a patient remote device in electronic communication with the thermal device. The thermal device includes heating elements and cooling elements arranged in a thermal array; a temperature sensor proximate to at least one heating element or cooling element; a printed circuit board; a microcontroller on the printed circuit board; a temperature reader on the printed circuit board and in communication with the at least one temperature sensor; a driver on the printed circuit board and in communication with the heating elements and the cooling elements; a non-volatile memory device on the printed circuit board; and a power supply.

### CROSS-REFERENCE TO RELATED APPLICATION(S)

The present application claims priority to and the benefit of U.S. Provisional Application No. 63/554,659, filed Feb. 16, 2024, the entire content of which is incorporated herein by reference.

### BACKGROUND

### 1. Field

The present disclosure relates to systems and methods for treating nervous system disorders and alleviating pain utilizing the thermal grill illusion.

### 2. Description of the Related Art

A variety of different devices have been developed to treat pain and nervous system disorders, including medications, spinal cord stimulation, deep brain stimulation, physical therapy, or surgery. However, many of these treatments are invasive and have the potential for complications or side effects.

The above information disclosed in this Background section is only for enhancement of understanding of the background of the invention and therefore it may contain information that does not constitute prior art.

### SUMMARY

The present disclosure relates to various embodiments of a system configured to alleviate pain. In one embodiment, the system is a thermal device and a patient remote device in electronic communication with the thermal device. The thermal device includes thermal elements, which are configured to provide heating and cooling, arranged in a thermal array; one or more temperature sensors proximate to at least one thermal element; a printed circuit board; a microcontroller on the printed circuit board; a temperature reader on the printed circuit board and in communication with at least one temperature sensor; a driver on the printed circuit board and in communication with the thermal elements; a non-volatile memory device on the printed circuit board; and a power supply.

The heating elements may be Peltier devices each having a warm side configured to face a user, and the cooling elements may be Peltier devices each having a cold side configured to face the user.

The heating elements may be resistive heating elements, and the plurality of cooling elements may be Peltier devices each having a cold side configured to face a user.

The heating elements and the cooling elements may be arranged in a checkerboard pattern.

The heating elements and the cooling elements may be arranged in alternating stripes.

The heating elements and the cooling elements may be arranged in alternating concentric rings.

The heating elements and the cooling elements may be arranged in alternating dots.

The non-volatile memory device may include computer-readable instructions which, when executed by the microcontroller in response to a signal from the patient remote, cause the at least one driver to simultaneously deliver power from the power supply to the heating elements and the cooling elements.

The non-volatile memory device may include computer-readable instructions which, when executed by the microcontroller in response to a signal from the patient remote, cause the at least one driver to alternate a direction of electric current from the power supply to the Peltier devices at a predefined switching frequency to switch each of the Peltier devices between hot and cold.

The non-volatile memory device may include computer-readable instructions which, when executed by the microcontroller in response to a temperature measurement from the at least one temperature sensor, automatically adjust current and/or voltage supplied to the heating elements or the cooling elements.

The system may include at least one light-emitting element configured to project light in between or through the heating elements and the cooling elements.

The at least one light-emitting element may be configured to emit light having a wavelength in a range from approximately 500 nm to approximately 1,200 nm.

The at least one light-emitting element may include at least one light-emitting diode.

The at least one light-emitting element may include at least one laser diode.

The system may include at least one vibration element.

The at least one vibration element may include an ultrasound element.

The at least one vibration element may include a low frequency vibration element configured to generate vibrations in a range from approximately 1 Hz to approximately 20,000 Hz.

The at least one vibration element may include a low frequency vibration element configured to generate vibrations in a range from approximately 5 Hz to approximately 1,000 Hz.

The thermal device further may include a wireless communication component on the printed circuit board, and the wireless communication component may be configured to communicate with the patient remote device via a wireless communication protocol.

The present disclosure also relates to various embodiments of a method of treating a patient suffering from pain. In one embodiment, the method includes placing a thermal device, which includes thermal elements (heating elements and cooling elements) arranged in a thermal array, on a portion of the patient's body, and activating the thermal elements.

The portion of the patient's body may be a lower back of the patient, and the activating of the heating elements and the cooling elements may reduce symptoms of back pain due to gate control theory.

The portion of the patient's body may be the patient's head or neck, and the activating of the heating elements and the cooling elements may stimulate the patient's trigeminal nerve and reduce symptoms of a migraine.

Activating the heating elements and the cooling elements may include simultaneously activating the heating elements and the cooling elements, and the heating elements and the cooling elements may be alternately arranged.

Activating the heating and the cooling elements may include alternating between operating the thermal device only in a heating mode and only in a cooling mode.

Activating the heating elements and the cooling elements may include activating the heating elements and the cooling elements in a first spatially alternating pattern for a first period of time, deactivating the cooling elements and the heating elements after expiration of the first period of time, activating the heating elements and the cooling elements, in a second spatially alternating pattern different than the first spatially alternating pattern, for a second period of time after the expiration of the first period of time, deactivating the heating elements and the cooling elements after expiration of the second period of time.

Each of the first period of time and the second period of time may be in a range from approximately 1 minute to approximately 20 minutes and activating in the first spatially alternating pattern and activating in the second spatially alternating pattern may be repeated substantially continuously.

The method may include irradiating, with light emitted from at least one light-emitting element of the thermal device, the portion of the patient's body.

The light may have a wavelength in the range from approximately 500 nm to approximately 1,200 nm wavelength.

The at least one light-emitting element may include at least light-emitting diode or at least one laser diode.

The method may include vibrating, with vibrations generated from at least one vibration element of the thermal device, and the vibration may cause an analgesic effect and increased blood circulation in the portion of the patient's body.

The vibrations may be in a range from approximately 1 Hz to approximately 20,000 Hz.

The at least vibration element may include an ultrasound element.

This summary is provided to introduce a selection of features and concepts of embodiments of the present disclosure that are further described below in the detailed description. This summary is not intended to identify key or essential features of the claimed subject matter, nor is it intended to be used in limiting the scope of the claimed subject matter. One or more of the described features may be combined with one or more other described features to provide a workable system or method.

### BRIEF DESCRIPTION OF THE DRAWINGS

The features and advantages of embodiments of the present disclosure will be better understood by reference to the following detailed description when considered in conjunction with the drawings. The drawings are not necessarily drawn to scale.

FIG. 1 is a schematic view of a system for treating pain applied to a lower back area of a patient according to one embodiment of the present disclosure;

FIG. 2 is a schematic block diagram of the embodiment of the system depicted in FIG. 1;

FIGS. 3A-3E depict a front view of a thermal device of the system of FIG. 2 according to various embodiments of the present disclosure;

FIG. 4 is a depiction of the principle of gate control theory; and

FIG. 5 is a flowchart illustrating tasks of a method of treating nervous system disorders and/or for alleviating pain.

### DETAILED DESCRIPTION

The terminology utilized herein is for the purpose of describing particular embodiments only and is not intended to be limiting of the present disclosure. As utilized herein, the singular forms “a”, “an” and “the” are intended to include the plural forms as well, unless the context clearly indicates otherwise. As utilized herein, the term “and/or” includes any and all combinations of one or more of the associated listed items.

It will be understood that, although the terms “first”, “second”, “third”, etc., may be utilized herein to describe one or more suitable elements, components, regions, and/or sections, these elements, components, regions, and/or sections should not be limited by these terms. These terms are only utilized to distinguish one element, component, region, layer or section from another element, component, region, layer or section. Thus, a first element, component, region, or section discussed could be termed a second element, component, region, or section, without departing from the spirit and scope of the present disclosure.

It will be understood that when an element is referred to as being “on”, “connected to”, “coupled to”, or “adjacent to” another element, it can be directly on, connected to, coupled to, or adjacent to the other element, or one or more intervening element(s) may be present. In contrast, when an element is referred to as being “directly on,” “directly connected to”, “directly coupled to”, or “immediately adjacent to” another element, there are no intervening elements present.

As utilized herein, the term “substantially” and similar terms are utilized as terms of approximation and not as terms of degree, and are intended to account for the inherent deviations in measured or calculated values that would be recognized by those of ordinary skill in the art. Also, the terms “about,” “approximately,” and similar terms, when utilized herein in connection with a numerical value or a numerical range, are inclusive of the stated value and refer to within an acceptable range of deviation for the particular value as determined by one of ordinary skill in the art, considering the measurement in question and the error associated with measurement of the particular quantity (e.g., the limitations of the measurement system).

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

Example embodiments of the present disclosure will now be described with reference to the accompanying drawings. In the drawings, the same or similar reference numerals refer to the same or similar elements throughout. As utilized herein, the utilize of the term “may,” when describing embodiments of the present disclosure, refers to “one or more embodiments of the present disclosure.”

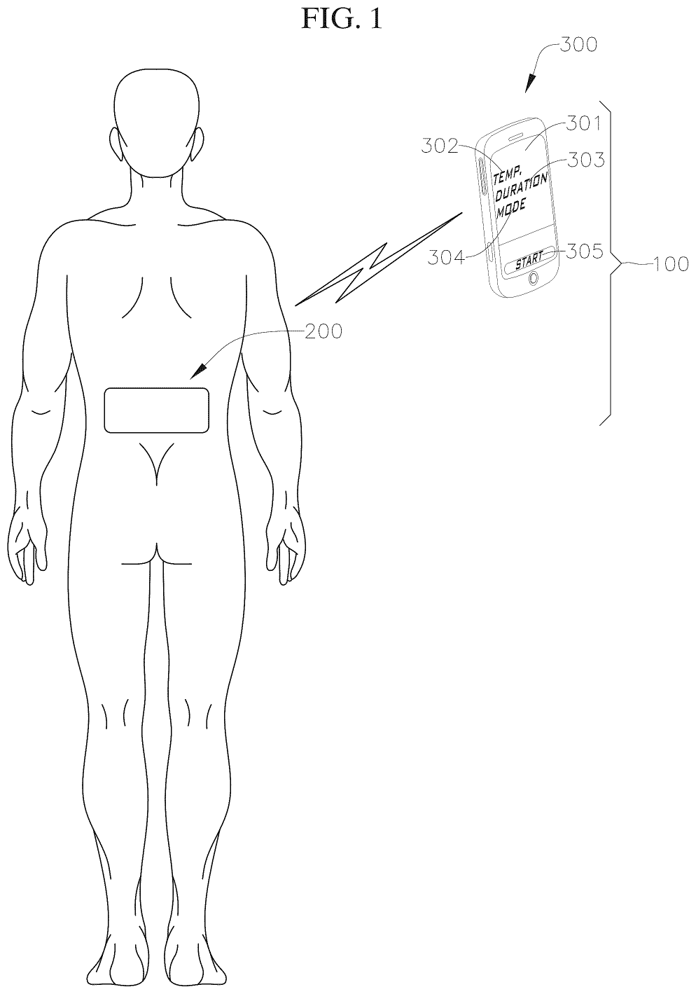

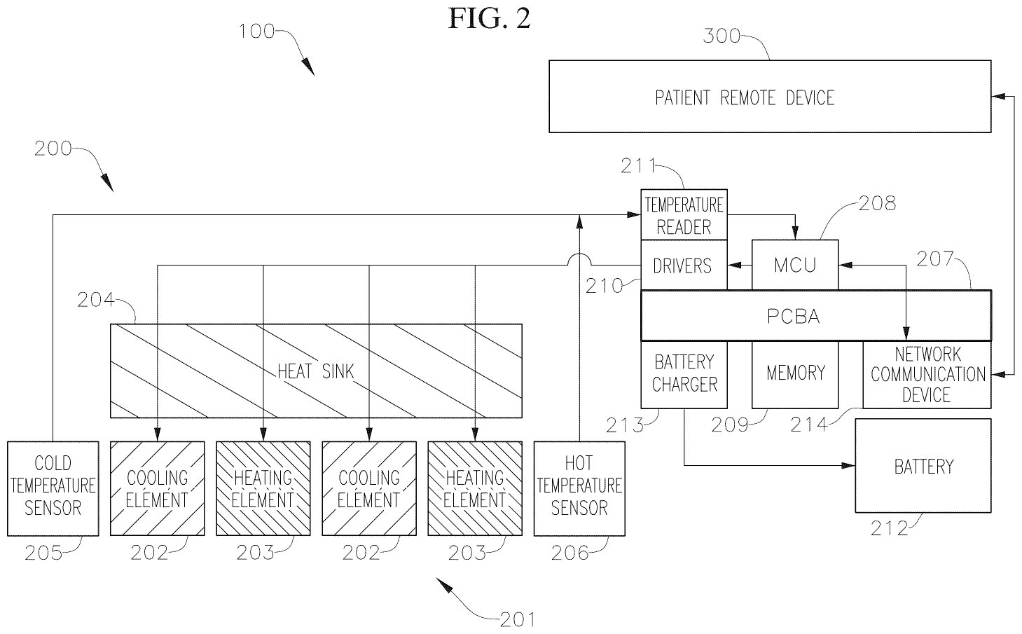

FIGS. 1-2 depict a system 100 for alleviating pain experienced by a patient according to one embodiment of the present disclosure. In the illustrated embodiment, the system 100 includes an external thermal device 200 and a patient remote (PR) device 300 in electronic communication with the external thermal device 200. In the illustrated embodiment, the thermal device includes a thermal array or grill 201 having a plurality of cooling elements or cooling devices 202 and a plurality of heating elements or heating devices 203. The cooling elements 202 and the heating elements 203 may also be referred to herein, either individually or collectively, as thermal elements. A Peltier device is a thermal control module or device that can provide either a “warming” or “cooling” effect on one surface of the device. By passing electric current through the Peltier device, it is possible to change the device surface temperature to become hotter or cooler and to maintain the desired surface temperature. When current is reversed through the Peltier device, the applicable device surface temperature can be switched from warming to cooling or from cooling to warming. In one or more embodiments, the thermal elements 202 may be Peltier devices that are configured to heat or cool as specified by (or according to) the programming of a controller. As used herein, the term “cold Peltier” device refers to a Peltier device having an applicable surface in a cooling state the term “hot Peltier” device refers to a Peltier device having an applicable surface in a heating or warming state. Each of the cold and hot Peltier devices 202, 203 includes a core sandwiched between a pair of thermally conductive plates (i.e., a hot plate and a cool plate) and a pair of electrical leads connected to the core. The core includes an alternating arrangement of p-type and n-type semiconductor elements. When a DC current is applied via the electrical leads, a heat flux is generated at the junctions between the p-type and the n-type semiconductor elements due to the Peltier effect. When the direction of the DC current is reversed, the heat flux is reversed, which causes the hot and cold sides of the Peltier devices 202, 203 to be switched. Additionally, changing the direction of the DC current enables pulsed heating and/or cooling at any desired rate. Changing the direction of the DC current also enables switching of the hot and cold elements in the thermal array or grill 201 arrangement to avoid habituation by the user. The switching may be performed at a predefined switching frequency (e.g., a symmetric waveform or an asymmetric waveform). In one or more embodiments, the heating and cooling elements may be any other suitable types or kinds or heating and cooling devices. For example, in one or more embodiments, the heating elements 203 may be resistive heating elements.

In one or more embodiments, the thermal array 201 may be flexible such that the thermal array 201 is configured to conform (or substantially conform) to different anatomical portions of a patient. For instance, in one or more embodiments, the thermal array 201 may be configured to conform (or substantially conform) to a portion of the patient's lower back to treat back pain, as illustrated in FIG. 1. In one or more embodiments, the thermal array 201 may be configured to conform (or substantially conform) to a portion of the patient's leg, shoulder, or neck.

In the illustrated embodiment, the external thermal device 200 also includes a heat sink 204 configured to dissipate heat from the thermal array 201. In the illustrated embodiment, the heat sink 204 is on a backside of the thermal array 201. When the external thermal device 200 is applied to the patient, the thermal array 201 faces the portion of the patient to which the thermal device 200 is applied, and the heat sink 204 faces away from the patient.

In the illustrated embodiment, the thermal device 200 also includes a first temperature sensor 205 connected to (or proximate to) one of the cooling devices 202 (e.g., one of the cold Peltier devices), and a second temperature sensor 206 connected to (or proximate to) one of the heating devices 203 (e.g., one of the hot Peltier devices). In one or more embodiments, the thermal device 200 may include one or more temperature sensors.

In the illustrated embodiment, the thermal device 200 also includes a printed circuit board (PCBA) 207, a microcontroller (MCU) 208 including at least one processor (or processor core) on the PCBA 207, a non-volatile memory device 209 (e.g., flash memory, or read-only memory (ROM), such as programmable read-only memory (PROM) or erasable programmable read-only memory (EPROM)), on the PCBA 207, one or more drivers 210 (e.g., one or more Peltier drivers) on the PCBA 207 and connected to the heating and cooling devices 202, 203, and one or more temperature readers 211 on the PCBA 207 and in electronic communication with (e.g., connected to) the first and second temperature sensors 205 and 206. The temperature sensors 205, 206 are configured to transmit or send the measured temperatures of the cooling and heating devices 202, 203, respectively, to the temperature reader 211, and the driver 210 is configured to adjust the signals (e.g., the DC voltage(s)) supplied to the cooling and heating devices 202, 203 based on the measured temperatures to achieve the desired cooling and heating temperatures provided by the cooling and heating devices 202, 203. In this manner, the thermal device 200 is configured to provide automatic closed-loop temperature control.

In the illustrated embodiment, the thermal device 200 also includes a power supply 212 (e.g., at least one battery, such as at least one secondary battery) and a battery charger 213 on the PCBA 207 and connected to the power supply 212. In one or more embodiments, the thermal device 200 may not include the power supply 212 (e.g., the battery) and thermal device 200 may instead include a power cable configured to be plugged into a wall outlet when in use. In one or more embodiments, the thermal device 200 may include both the power supply 212 (e.g., the battery) and the power cable.

The thermal device 200 also includes a wireless communication component or a network communication device (circuit) 214 (e.g., an antenna, such as a transceiver or a receiver and a transmitter) on the PCBA 207 that is configured to wirelessly communicate with the PR device 300. Wireless links may include Bluetooth™, Bluetooth Low Energy or other protocols. In one or more embodiments, the wireless communication protocol may include an authentication and encryption protocol to protect patient data. In the illustrated embodiment, the temperature reader(s) 211, the driver(s) 210, the non-volatile memory device 209, and the wireless communication component 214 are connected to each other over the MCU 208. In one or more embodiments, the PR device 300 may be connected to the thermal device 200 via one or more wires (e.g., a cable) and the thermal device 200 may not include the wireless communication component 214.

The term “processor” is utilized herein to include any combination of hardware, firmware, memory, and software, employed to process data or digital signals. Analog inputs and/or outputs to the processor may also be employed. The hardware of a processor may include, for example, a microcontroller, application specific integrated circuits (ASICs), general purpose or special purpose central processors (CPUs), digital signal processors (DSPs), graphics processors (GPUs), analog to digital converters, digital to analog converters, and programmable logic devices such as field programmable gate arrays (FPGAs). In a processor, as utilized herein, each function is performed either by hardware configured, i.e., hard-wired, to perform that function, or by more general-purpose hardware, such as a CPU, configured to execute instructions stored in a non-transitory storage medium or memory. A processor may contain two or more processors, for example, a processor may include two processors, an FPGA and a CPU, interconnected on the PCBA 207.

The PR device 300 may be any suitable electronic device, such as a smartphone. Additionally, in one or more embodiments, the PR device 300 may display one or more parameters for controlling operation of the thermal device 200. In one or more embodiments, a display 301 of the PR device 300 may display a graphical user interface (GUI) including one or more buttons, sliders, or menus for controlling one or more parameters of the thermal device 200. For instance, in one or more embodiments, the GUI displayed on the PR device 300 may include a field 302 for entering the operating temperatures (or range of temperatures) of the cooling and heating devices 202, 203, a field 303 for entering the duration of operation of the thermal device 200, a field 304 for entering the operating mode of the thermal device 200, and a button 305 for activating or deactivating the thermal device 200.

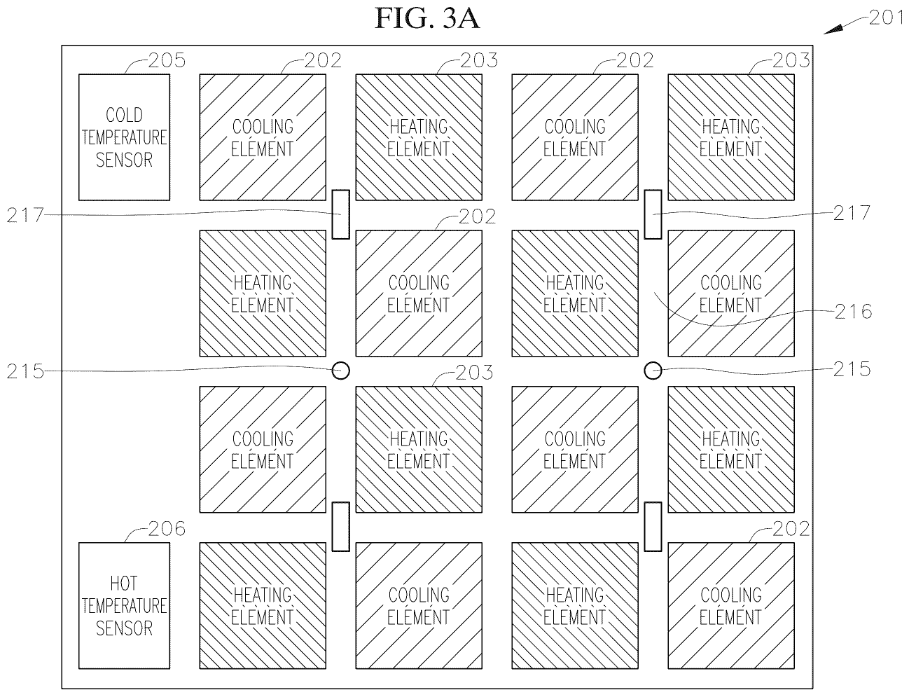

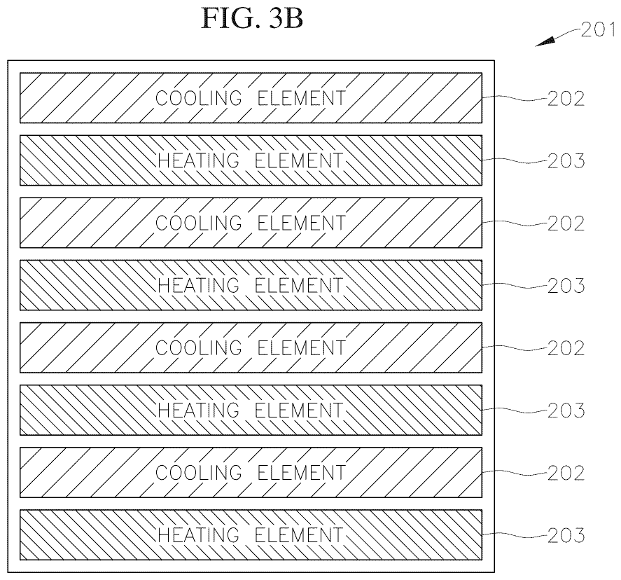

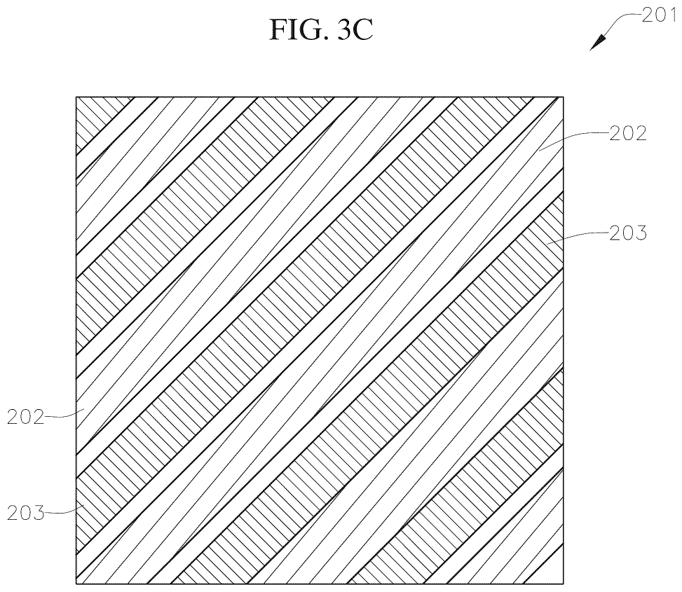

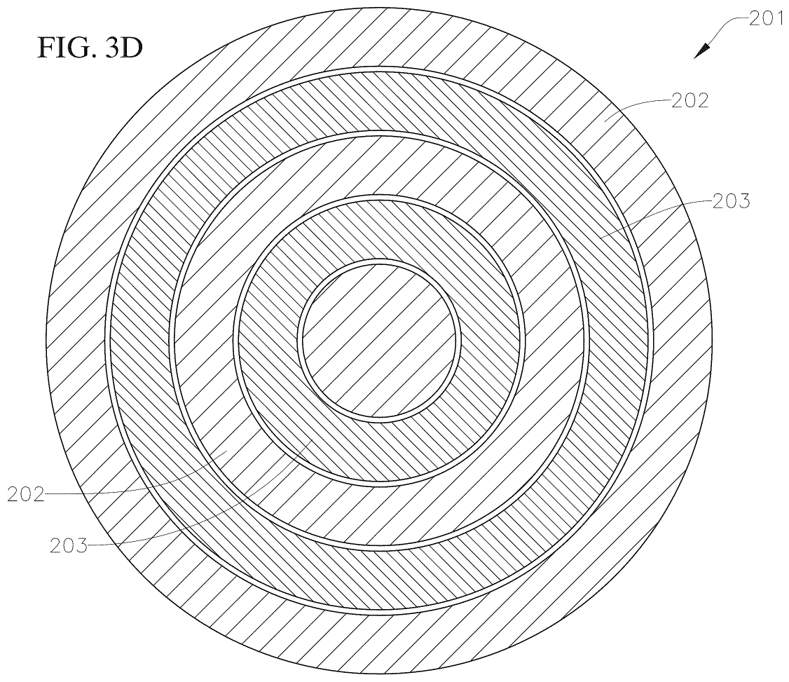

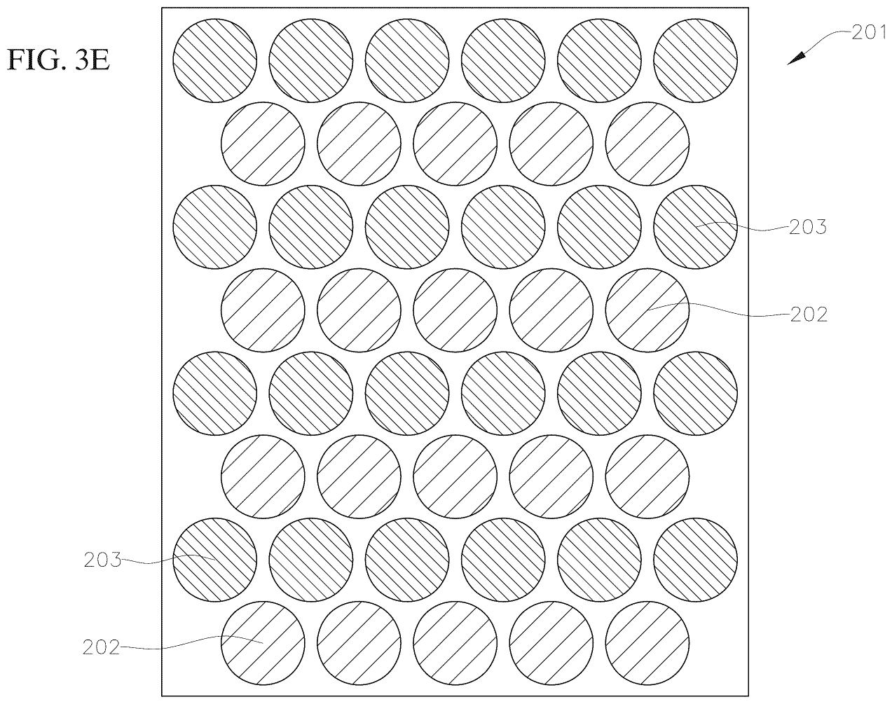

FIGS. 3A-3E depict different configurations and arrangements of the cooling and heating devices 202, 203 (e.g., the hot and cold Peltier devices which may be reversed from cold-to-hot and hot-to-cold) according to various embodiments of the present disclosure. In the embodiment illustrated in FIG. 3A, the cooling and heating devices 202, 203 are square-shaped and are alternately arranged in a grid pattern including a series of rows and a series of columns (i.e., a checkerboard pattern). In the embodiment illustrated in FIG. 3B, the cooling and heating devices 202, 203 are rectangle-shaped and are alternately arranged in a series of alternating parallel (or substantially parallel) stripes. Although in the embodiment illustrated in FIG. 3B the cooling and heating devices 202, 203 are arranged vertically, in one or more embodiments the cooling and heating devices 202, 203 may be arranged in any other suitable orientation, such as horizontally. In the embodiment illustrated in FIG. 3C, the cooling and heating devices 202, 203 are trapezoid-shaped and are arranged in a series of alternating slanted (e.g., diagonal) stripes. In the embodiment illustrated in FIG. 3D, the cooling and heating devices 202, 203 are ring-shaped (i.e., annular) and are arranged in a series of concentric (or substantially concentric) rings. In the embodiment illustrated in FIG. 3E, the cooling and heating devices 202, 203 are dot-shaped (e.g., circular) and are arranged in a series of rows. Each row includes a plurality of heating elements 203 or a plurality of cooling elements 202, and the rows that include the heating elements 203 are alternately arranged with the rows that include the cooling elements 202. Although in the illustrated embodiment the dot-shaped cooling and heating elements 202, 203 are arranged in a series of horizontal rows, in one or more embodiments the dot-shaped cooling and heating elements 202, 203 may be arranged in any other suitable configuration. In one or more embodiments, the thermal device 200 may have a width in a range from approximately 2 inches to approximately 24 inches, and a height in a range from approximately 2 inches to approximately 18 inches (e.g., in one embodiment, the thermal device 200 may have a width of approximately 8 inches and a height of approximately 6 inches). In one or more embodiments, the thermal device 200 may be as small as approximately 2 inches×approximately 2 inches, and the thermal device 200 may be as large as approximately 18 inches×approximately 24 inches. Additionally, in one or more embodiments, the thermal device 200 may have a rectangular shape, a square shape, a circular shape, or any other shape suitable for the anatomical area on which the thermal device 200 is intended to be applied. Furthermore, although in one or more embodiments, the thermal device 200 includes a plurality of cooling elements 202 and a plurality of heating elements 203, in one or more embodiments the thermal device 200 may include a single thermal element that can be powered to alternately heat and cool over time, e.g., a Peltier device. While a single thermal element cannot induce the Thermal Grill Effect, the thermal device 200 would still be configured to provide the other benefits of heating and cooling in a controlled, timed, and alternating manner. In one or more embodiments, the thermal device 200 may include a single cooling element 202 and a single heating element 203. It should be understood that, in any of these embodiments, the heat and cooling may be interchanged, or pulsed from hot to cold and from cold to hot.

In one or more embodiments, the thermal device 200 may include one or more light-emitting elements 215 (e.g., one or more light-emitting diodes (LEDs) or laser diodes) in one or more of the gaps 216 between adjacent cooling and heating elements 202, 203 (e.g., a gap 216 between one cooling element 202 and an adjacent heating element 203). In one or more embodiments, the light-emitting elements 215 may be configured to emit light having a wavelength in the range from approximately 500 nm to approximately 1,200 nm. The light emitted from the light-emitting elements 215 is configured to promote healing. In one or more embodiments, the cooling and heating elements 202, 203 may be substantially transparent to permit the transmission of light from the light-emitting elements through the majority of the area of the thermal device 200. In an embodiment in which the cooling and heating elements 202, 203 are Peltier heating/cooling elements, the outer sheets of material, typically opaque alumina, could be made of a clear material, such as single-crystal alumina (sapphire) or a relatively thermally conductive glass. Additionally, in one or more embodiments, the P-N junctions inside the Peltier devices may not be transparent, but the P-N junctions may be reduced in number and spaced further apart to allow light to pass around them.

In one or more embodiments, the thermal device 200 may include one or more vibration elements 217 (e.g., one or more ultrasound elements or low frequency vibration elements) in one or more of the gaps 216 between adjacent cooling and heating elements 202, 203 (e.g., a gap 216 between one cooling element 202 and an adjacent heating element 203). In one or more embodiments, the vibration elements 217 may be configured to generate vibrations in a range from approximately 1 Hz to approximately 20,000 Hz. In one or more embodiments, the vibration elements 217 may be configured to generate vibrations in the ultrasonic range above approximately 20,000 Hz. In one or more embodiments, the vibration elements 217 may be configured to generate vibrations in a range from approximately 5 Hz to approximately 1,000 Hz. In one or more embodiments, the vibration elements 217 may be electronic muscle stimulation (EMS) devices. The EMS devices may be configured to generate vibrations in a range from approximately 1 Hz to approximately 150 Hz. The one or more vibration elements 217 are configured to provide an analgesic effect and promote blood circulation in the patient to promote healing.

In operation, the PR device 300 is configured to send (e.g., transmit) a signal to the thermal device 200 to operate the cooling and heating elements 202, 203. The signal may be transmitted, for example, in response to a user entering a command (e.g., pushing button 304) on the GUI displayed on the PR device 300. In one or more embodiments, the signal from the PR device 300 may be received by the wireless communication component 214. In one or more embodiments, the signal from the PR device 300 may be received by a wire or cable connected to the thermal device 200. In one or more embodiments, the non-volatile memory device 209 of the thermal device 200 includes instructions which, when executed by the MCU 208 in response to receipt of the signal from the PR device 300, cause the driver(s) 210 to deliver DC current(s) from the power supply 212 to the cooling and heating elements 202, 203. The DC current causes the heating elements 203 to generate heat at the side adjacent to the skin and causes the cooling elements 202 to remove heat from the side adjacent to the skin.

Together, the heat generated by the heating elements 203 and the thermal dissipation caused by the cooling elements 202 causes a thermal grill effect (i.e., a thermal grill illusion) when the thermal device 200 is applied to a patient. The thermal grill illusion refers to a sensory illusion in which the interlacing of hot and cold elements causes a pain sensation in a healthy subject without a noxious stimulus (i.e., the thermal grill illusion activates a region of the brain associated with noxious thermal stimuli to generate a pain sensation).

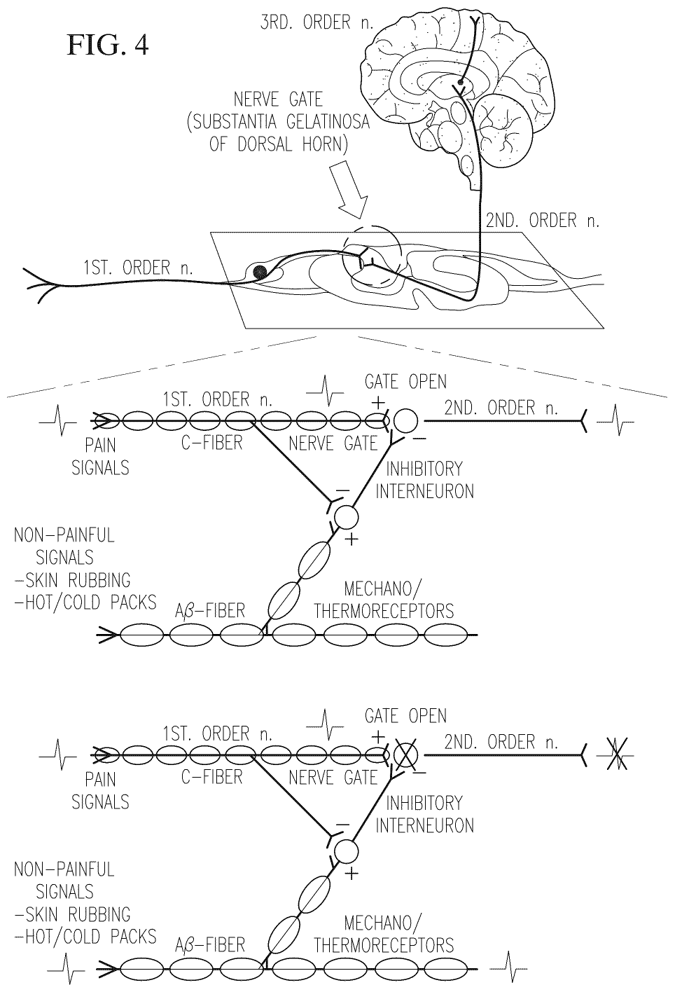

The thermal grill effect or illusion generated by the thermal device 200 may be utilized to alleviate or desensitize the patient to pain due to gate control theory. Similarly, light and vibration may be utilized to alleviate or desensitize the patient to pain due to gate control theory. As illustrated in FIG. 4, gate control theory states that spinal nerves act as a gate that can be open to let pain signals travel to the brain or closed to prevent pain signals from reaching the brain. As illustrated in FIG. 4, innocuous stimuli are transmitted along Aβ-fibers and pain signals are transmitted along C-fibers. Inhibitory interneurons in the superficial dorsal horn, which may be activated by the innocuous signal transmitted through the Aβ-fibers, may inhibit the transmission of pain signals through the C-fibers. The illusory pain sensation (i.e., the thermal grill illusion) that the system 100 causes the user to experience may be transmitted along the Aβ-fibers and may activate the inhibitory interneurons, thereby inhibiting the pain signals passing through the C-fibers. In this manner, the system 100 is configured to alleviate pain, including chronic pain (e.g., back pain, joint pain, or chronic migraines) or acute pain (e.g., migraines, a strained muscle, or a post-operative surgical site)

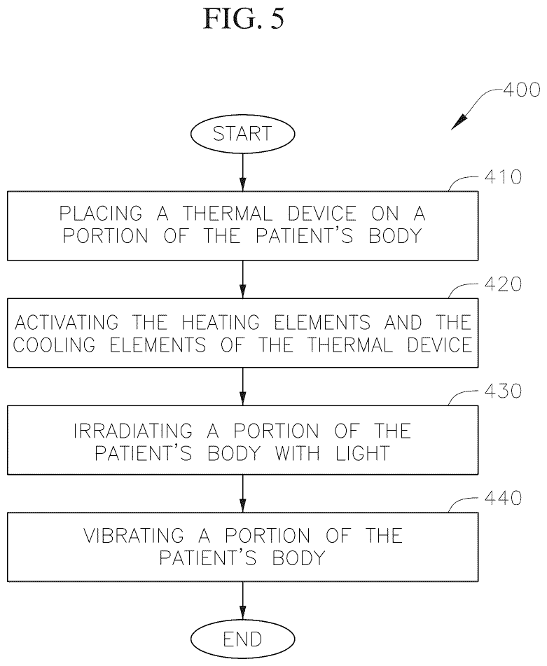

FIG. 5 is a flowchart illustrating tasks of a method 400 of treating a patient suffering from pain according to one embodiment of the present disclosure. In the illustrated embodiment, the method 400 includes a task 410 of placing a thermal device on a portion of the patient's body that is experiencing pain (e.g., acute pain, such as a strained muscle or a post-operative surgical site; or chronic pain, such as back pain or joint pain). In one or more embodiments, the task 410 may include placing the thermal device on the patient's lower back, upper back, leg, shoulder, or neck. The thermal device may be the same as or similar to the embodiment of the thermal device 200 depicted in FIGS. 1-2.

The method 400 also includes a task 420 of activating the thermal elements (e.g., activating the Peltier devices) of the thermal device. In one or more embodiments, the task 420 may include simultaneously (or substantially simultaneously) activating all the thermal elements. Simultaneously activating the thermal elements is configured to cause a thermal grill effect by causing adjacent thermal elements to be of opposite temperature (i.e., warm/cold/warm/cold, inducing the thermal grill illusion) such that the patient experiences a slightly painful sensation even though there is no noxious (pain) stimulus. As described above with reference to FIG. 4, according to the gate theory of pain, the illusory pain sensation perceived by the patient is configured to alleviate the chronic and/or acute pain experienced by the patient. In one or more embodiments, the task 410 may include applying the thermal device to a lower back area of the patient, and the task 420 of activating the heating and cooling elements may reduce the lower back pain experienced by the patient. In one or more embodiments, the task 410 may include applying the thermal device to the back of the patient's head or neck, and the task 420 of activating the heating and cooling elements may stimulate the patient's 5th cranial (trigeminal) nerve and thereby reduce the symptoms of a migraine.

In one or more embodiments, the task 420 may include alternately activating all the thermal elements in synchrony to be either hot or cold sequentially over time to achieve the application of alternating hot and cold compresses to a portion of the patient's body, which may be utilized to relieve muscle strain or soreness. For instance, in one or more embodiments, the task 420 may include activating all or some of the thermal elements, e.g., Peltier devices, in a cooling mode for a first period of time (e.g., in a range from approximately 1 minute to approximately 20 minutes) and then activating those same thermal elements (previously cooling elements) in a heating mode for a second period of time (e.g., in a range from approximately 1 minute to approximately 20 minutes) after the expiration of the first period of time and deactivating the heating after expiration of the second period of time. The second period of time may be the same as (or substantially the same) the first period of time, or the second period of time may be different than the first period of time. This process of alternately activating the thermal elements in a heating and cooling mode may be repeated for a number of cycles.

In one or more embodiments, the task 420 may include activating the heating elements and the cooling elements in a first spatially alternating pattern (e.g., warm/cold/warm/cold) for a first period of time (e.g., in a range from approximately 1 minute to approximately 20 minutes), deactivating the cooling elements and the heating elements after expiration of the first period of time, activating the heating elements and the cooling elements in a second spatially alternating pattern different than the first spatially alternating pattern for a second period of time (e.g., in a range from approximately 1 minute to approximately 20 minutes) after the expiration of the first period of time, and deactivating the heating elements and the cooling elements after expiration of the second period of time. In one embodiment, the thermal elements that are warm would be become cold, and the thermal elements that are cold would become warm and, after a time, vice versa, back and forth with a switch interval in the range from approximately 1 to approximately 5 minutes. This switching methodology of the therapy prevents habituation to the thermal grill illusion, thereby maximizing (or at least increasing) its therapeutic effect.

In one or more embodiments, the method 400 may include a task 430 of irradiating the portion of the patient's body with light emitted from at least one light-emitting element of the thermal device. The task 430 of irradiating the portion of the patient's body with light is configured to accelerate healing, promote blood flow, and reduce inflammation. In one or more embodiments, the light-emitting element may be a light-emitting diode (LED) or a laser diode. In one or more embodiments, the task 430 may include irradiating the portion of the patient's body with light having a wavelength in a range from approximately 500 nm to approximately 1,200 nm. In one or more embodiments, the method 400 may not include the task 430 of irradiating the portion of the patient's body with light.

In one or more embodiments, the method 400 may include a task 440 of vibrating the portion of the patient's body with vibrations generated from at least one vibration element of the thermal device. Vibrating the portion of the patient's body is configured to cause an analgesic effect and increase blood circulation in the portion of the patient's body. The vibrations applied in this task 440 may be generated from an ultrasound element or a low frequency vibration element. In one or more embodiments, the task 440 may include vibrating the portion of the patient's body with vibrations in a range from approximately 1 Hz to approximately 20,000 Hz, such as in a range from approximately 10 Hz to approximately 1,000 Hz. In one or more embodiments, the method 400 may or may not include the task 440 of vibrating the portion of the patient's body.

The system, any other relevant devices or components, and the method according to embodiments of the present disclosure described herein may be implemented utilizing any suitable hardware, firmware (e.g., an application-specific integrated circuit), software, or a combination of software, firmware, and hardware. For example, the one or more suitable components of the system may be formed on one integrated circuit (IC) chip or on separate IC chips. Further, the one or more suitable components of the system may be implemented on a flexible printed circuit film, a tape carrier package (TCP), a printed circuit board (PCB), or formed on one substrate. Further, the one or more suitable components of the system may be a process or thread, running on one or more processors, in one or more computing devices, executing computer program instructions and interacting with other system components for performing the one or more suitable functionalities described herein. The computer program instructions are stored in a memory which may be implemented in a computing device utilizing a standard memory device, such as, for example, a random access memory (RAM). The computer program instructions may also be stored in other non-transitory computer readable media such as, for example, a flash drive, and/or the like. Also, a person of skill in the art should recognize that the functionality of one or more suitable computing devices may be combined or integrated into a single computing device, or the functionality of a particular computing device may be distributed across one or more other computing devices without departing from the scope of the example embodiments of the present disclosure.

Although some embodiments of the present disclosure have been disclosed herein, the present disclosure is not limited thereto, and the scope of the present disclosure is defined by the appended claims and equivalents thereof.

What is claimed is:

1. A system configured to alleviate pain, the system comprising:
a thermal device comprising:
a plurality of heating elements and a plurality of cooling elements arranged in a thermal array;
at least one temperature sensor proximate to at least one of the plurality of heating elements and the plurality of cooling elements;
a printed circuit board;
a microcontroller on the printed circuit board;
at least one temperature reader on the printed circuit board and in communication with the at least one temperature sensor;
at least one driver on the printed circuit board and in communication with the plurality of heating elements and the plurality of cooling elements;
a non-volatile memory device on the printed circuit board; and
a power supply; and
a patient remote device in electronic communication with the thermal device.

2. The system of claim 1, wherein the plurality of heating elements is a plurality of Peltier devices each comprising a warm side configured to face a user, and wherein the plurality of cooling elements is a plurality of Peltier devices each comprising a cold side configured to face the user.

3. The system of claim 1, wherein the plurality of heating elements is a plurality of resistive heating elements, and wherein the plurality of cooling elements is a plurality of Peltier devices each comprising a cold side configured to face a user.

4. The system of claim 1, wherein the plurality of heating elements and the plurality of cooling elements are arranged in a checkerboard pattern.

5. The system of claim 1, wherein the plurality of heating elements and the plurality of cooling elements are arranged in alternating stripes.

6. The system of claim 1, wherein the plurality of heating elements and the plurality of cooling elements are arranged in alternating concentric rings.

7. The system of claim 1, wherein the plurality of heating elements and the plurality of cooling elements are arranged in alternating dots.

8. The system of claim 1, wherein the non-volatile memory device includes computer-readable instructions which, when executed by the microcontroller in response to a signal from the patient remote, cause the at least one driver to simultaneously deliver power from the power supply to the plurality of heating elements and the plurality of cooling elements.

9. The system of claim 2, wherein the non-volatile memory device includes computer-readable instructions which, when executed by the microcontroller in response to a signal from the patient remote, cause the at least one driver to alternate a direction of electric current from the power supply to the plurality of Peltier devices at a predefined switching frequency to switch each of the plurality of Peltier devices between hot and cold.

10. The system of claim 1, wherein the non-volatile memory device includes computer-readable instructions which, when executed by the microcontroller in response to a temperature measurement from the at least one temperature sensor, automatically adjust current and/or voltage supplied to the plurality of heating elements or the plurality of cooling elements.

11. The system of claim 1, further comprising at least one light-emitting element configured to project light in between or through the plurality of heating elements and the plurality of cooling elements.

12. The system of claim 11, wherein the at least one light-emitting element is configured to emit light having a wavelength in a range from approximately 500 nm to approximately 1,200 nm.

13. The system of claim 11, wherein the at least one light-emitting element comprises at least one light-emitting diode.

14. The system of claim 11, wherein the at least one light-emitting element comprises at least one laser diode.

15. The system of claim 1, further comprising at least one vibration element.

16. The system of claim 15, wherein the at least one vibration element comprises an ultrasound element.

17. The system of claim 15, wherein the at least one vibration element comprises a low frequency vibration element configured to generate vibrations in a range from approximately 1 Hz to approximately 20,000 Hz.

18. The system of claim 15, wherein the at least one vibration element comprises a low frequency vibration element configured to generate vibrations in a range from approximately 5 Hz to approximately 1,000 Hz.

19. The system of claim 1, wherein the thermal device further comprises a wireless communication component on the printed circuit board, and wherein the wireless communication component is configured to communicate with the patient remote device via a wireless communication protocol.

20. A method of treating a patient suffering from pain, the method comprising:
placing a thermal device on a portion of the patient's body, the thermal device comprising a plurality of heating elements and a plurality of cooling elements arranged in a thermal array; and
activating the plurality of heating elements and the plurality of cooling elements.

21. The method of claim 20, wherein the portion of the patient's body is a lower back of the patient, and wherein the activating of the plurality of heating elements and the plurality of cooling elements reduces symptoms of back pain due to gate control theory.

22. The method of claim 20, wherein the portion of the patient's body is the patient's head or neck, and wherein the activating of the plurality of heating elements and the plurality of cooling elements stimulates the patient's trigeminal nerve and reduces symptoms of a migraine.

23. The method of claim 20, wherein the activating the plurality of heating elements and the plurality of cooling elements comprises simultaneously activating the plurality of heating elements and the plurality of cooling elements, and wherein the plurality of heating elements and the plurality of cooling elements are alternately arranged.

24. The method of claim 20, wherein the activating the plurality of heating and the plurality of cooling elements comprises alternating between operating the thermal device only in a heating mode and only in a cooling mode.

25. The method of claim 20, wherein the activating the plurality of heating elements and the plurality of cooling elements comprises:
activating the plurality of heating elements and the plurality of cooling elements in a first spatially alternating pattern for a first period of time;
deactivating the plurality of cooling elements and the plurality of heating elements after expiration of the first period of time;
activating the plurality of heating elements and the plurality of cooling elements, in a second spatially alternating pattern different than the first spatially alternating pattern, for a second period of time after the expiration of the first period of time; and
deactivating the plurality of heating elements and the plurality of cooling elements after expiration of the second period of time.

26. The method of claim 25, wherein each of the first period of time and the second period of time is in a range from approximately 1 minute to approximately 20 minutes and wherein the activating in the first spatially alternating pattern and the activating in the second spatially alternating pattern are repeated substantially continuously.

27. The method of claim 20, further comprising irradiating, with light emitted from at least one light-emitting element of the thermal device, the portion of the patient's body.

28. The method of claim 27, wherein the light has a wavelength in the range from approximately 500 nm to approximately 1,200 nm wavelength.

29. The method of claim 27, wherein the at least one light-emitting element comprises at least light-emitting diode or at least one laser diode.

30. The method of claim 20, further comprising vibrating, with vibrations generated from at least one vibration element of the thermal device, and wherein the vibration causes an analgesic effect and increased blood circulation in the portion of the patient's body.

31. The method of claim 30, wherein the vibrations are in a range from approximately 1 Hz to approximately 20,000 Hz.

32. The method of claim 30, wherein the at least vibration element comprises an ultrasound element.
